(19) United States
(12) Patent Application Publication
(10) Pub. No.: US 2026/0098001 A1
(43) Pub. Date: Apr. 9, 2026

(54) METHODS AND SYSTEMS FOR RENEWABLE ENERGY CONVERSION AND STORAGE

(71) Applicant: Aclara Technologies LLC, St. Louis, MO (US)

(72) Inventor: David W. Rieken, O’Fallon, MO (US)

(21) Appl. No.: 19/353,208

(22) Filed: Oct. 8, 2025

Related U.S. Application Data
(60) Provisional application No. 63/704,630, filed on Oct. 8, 2024.

(51) Int. Cl. C07C 1/12; C25B 1/04; C25B 15/08; H02S 10/20

(52) U.S. Cl. CPC C07C 1/12; C25B 1/04; C25B 15/081; H02S 10/20

(57) ABSTRACT

Disclosed herein are systems and methods for converting and storing renewable energy. An example system includes a source of electrical energy, an electrolyzer unit, a source of carbon dioxide, and a reactor unit that can produce synthetic methane in a Sabatier reaction. An example method includes using electrical energy to electrolyze water to produce hydrogen and oxygen, and reacting the hydrogen with carbon dioxide to produce synthetic methane in a Sabatier reaction.

### CROSS-REFERENCE TO RELATED APPLICATIONS

This application claims priority to U.S. Provisional Patent Application No. 63/704,630 filed on Oct. 8, 2024, which is incorporated fully herein by reference.

### TECHNICAL FIELD

The present disclosure relates to systems, methods, and techniques for converting and storing renewable energy.

### INTRODUCTION

The increasing adoption of solar energy, driven by its decreasing costs and environmental benefits, presents significant challenges in managing surplus electricity and ensuring reliable power supply. Solar panels produce electricity only when the sun is shining, leading to periods of surplus generation during the day and energy shortages at night or during cloudy weather. This intermittent supply of solar energy, combined with the reverse power flow into the grid during periods of low demand, puts a strain on distribution networks, leading to inefficiencies and potential instability in the grid.

To effectively integrate large-scale solar energy into the power grid, there is a need for solutions that can store excess electricity when generation exceeds demand and release it when needed, without relying solely on fossil fuels or creating additional strain on existing infrastructure. Present strategies to meet this problem include batteries and synthetic hydrogen. However, batteries suffer from high-cost and limited capacity, while synthetic hydrogen faces infrastructure issues of storage and distribution, as well as overall limited compatibility with existing natural gas networks. While both batteries and synthetic hydrogen have potential in specific contexts, their economic and infrastructural limitations make them less viable as scalable solutions for managing surplus solar energy.

### SUMMARY

In one aspect, described herein are systems for producing synthetic methane including: a source of electrical energy; an electrolyzer unit electrically coupled to the source of electrical energy, the electrolyzer unit configured to electrolyze water to form hydrogen and oxygen; a source of carbon dioxide; and a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction.

In another aspect, described herein are systems for producing synthetic methane including: a source of electrical energy; an electrolyzer unit electrically coupled to the source of electrical energy, the electrolyzer unit configured to electrolyze water to form hydrogen and oxygen; a source of carbon dioxide; a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction; a storage unit coupled to the reactor unit via a gas network, the storage unit configured to store synthetic methane produced by the reactor unit; and a combustion unit coupled to the storage unit and the reactor unit via the gas network, the combustion unit configured to burn methane to generate electricity.

In another aspect, described herein are methods for producing synthetic methane, the method including: using an electrical energy to electrolyze water to produce hydrogen and oxygen; and reacting the hydrogen with carbon dioxide to produce synthetic methane in a Sabatier reaction.

### BRIEF DESCRIPTION OF THE DRAWINGS

FIG. 1 is a schematic of an example system for converting and storing renewable energy.

FIG. 2 is a flowchart of an example method for producing synthetic methane.

### DETAILED DESCRIPTION

Unlike battery storage, which remains expensive and limited in capacity, the present disclosure provides a scalable and economical solution by converting surplus solar energy into synthetic methane. The use of existing natural gas infrastructure for storage and distribution further reduces costs. Synthetic methane can be integrated into the current natural gas network, eliminating the need for expensive and complex infrastructure upgrades required for alternatives like hydrogen. This makes it immediately deployable at scale, leveraging the vast, established gas distribution system. In addition, unlike batteries, which degrade over time and have limited storage capacity, synthetic methane can be stored for extended periods without loss of energy, making it ideal for balancing intermittent solar supply over days, weeks, or even seasons. The disclosed process is carbon neutral, as the carbon dioxide used to synthesize methane can be re-released when the methane is burned, resulting in no net increase in CO₂ emissions. This aligns with global efforts to reduce greenhouse gases while providing reliable energy. Finally, by converting surplus solar energy into methane, the present disclosure helps manage reverse power flow and smooth out energy supply fluctuations. This can ensure a more stable and reliable grid, even in periods of high solar generation or low demand.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed technology. All publications, patent applications, patents and other references mentioned herein are incorporated by reference in their entirety.

Before any embodiments are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. The terms “mounted,” “connected,” and “coupled” are used broadly and encompass both direct and indirect mounting, connecting, and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 104^{th} Ed., inside cover, and specific functional groups are defined as described therein.

### 2. Example Systems & Methods

The present disclosure addresses the challenge of storing surplus solar energy and ensuring its availability when needed, without relying on expensive and limited battery systems or the infrastructure required for synthetic hydrogen. The disclosed system utilizes synthetic methane as a medium for energy storage, providing a scalable and cost-effective solution for integrating large-scale solar energy into the grid.

The system and methods disclosed herein can convert surplus electricity generated by solar panels into synthetic methane via a two-step process. First, excess electricity can be used to power the electrolysis of water, producing hydrogen and oxygen. Next, the hydrogen can be combined with carbon dioxide in a Sabatier reaction, resulting in methane and water. The produced methane can be injected directly into the existing natural gas network or stored on-site. When solar availability is low and energy demand is high, the stored methane can be burned to generate electricity, effectively acting as a renewable energy buffer.

Synthetic methane can leverage the existing natural gas infrastructure, making it a more feasible and cost-effective solution. Additionally, the disclosed systems and methods can achieve carbon neutrality, e.g., the same amount of carbon dioxide used in the methane production process is released during combustion, ensuring no net increase in carbon emissions. By utilizing synthetic methane, the present disclosure offers a novel approach to long-term energy storage, capable of meeting the intermittent supply of solar power and stabilizing the grid.

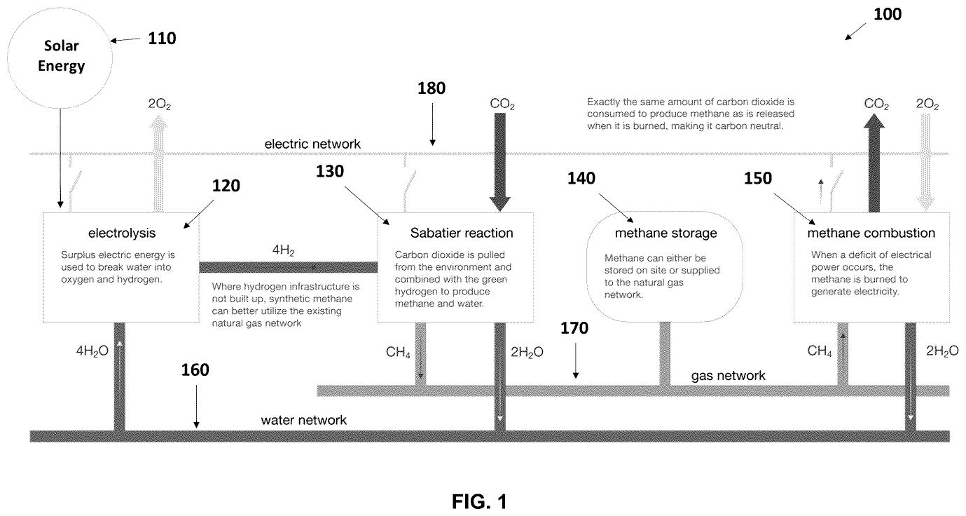

FIG. 1 illustrates an example system 100 for producing synthetic methane. Synthetic methane is a renewable gas (i.e., not extracted from natural gas) that can be produced using an industrial process of methanation, e.g., reacting carbon dioxide with hydrogen to provide synthetic methane. In the illustrated embodiment, the system 100 includes a source of electrical energy 110, an electrolyzer unit 120, a reactor unit 130, a storage unit 140, a combustion unit 150, a water network 160, a gas network 170, and an electric network 180.

The source of electrical energy 110 can be generated from a renewable energy, which as shown in FIG. 1 can be solar energy. Other examples of renewable energy include wind, wave, and tidal sources. In some embodiments, the source of electrical energy 110 is generated from solar energy. The source of electrical energy 110 can be in the form of a solar panel. In some embodiments, the source of electrical energy 110 is a plurality of solar panels, e.g., spread over a large area, such as in a high distributed energy resource (DER)-penetration substation. Such substations could shift from net loads to net sources during periods of sunshine.

The source of electrical energy 110 can be coupled (e.g., electrically coupled) to an electrolyzer unit 120 via the electric network 180. In some embodiments, the source of electrical energy 110 can be reversibly coupled and uncoupled to the electrolyzer unit 120. For example, if there is a surplus of electrical energy, the source of electrical energy 110 can be coupled to the electrolyzer unit 120 to create and store synthetic methane. Conversely, if there is not a surplus of electrical energy, the source of electrical energy 110 can be uncoupled from the electrolyzer unit 120. Reversible coupling can be done through a switch that is, e.g., controlled by an intelligent control system. During periods of reverse power flow, it can be turned on, functioning as a load. This can stop the power flow from the substation onto the transmission network.

The electrolyzer unit 120 can be configured to use the electrical energy 110 to electrolyze water to form hydrogen (H₂) and oxygen (O₂). Or in other words, the electrolyzer unit 120 can provide a suitable environment for electrolysis of water to take place. For example, an electric current can pass through the water using two electrodes (e.g., anode and cathode). At the cathode, hydrogen can be generated. At the anode, oxygen gas can be released. The water source or water network 160 can be coupled to the electrolyzer unit 120. The water source or water network 160 can be a finite reservoir of water because in principle the system 100 can theoretically produce the same amount of water as it consumes. Water can come from a water utility. Water can also come from a river or a lake. However, in some embodiments, the water is not captured from the atmosphere, such as from air moisture. Following electrolysis of the water, the produced hydrogen can be fed or received by the reactor unit 130. As such, the electrolyzer unit 120 can be coupled to the reactor unit 130. In some embodiments, the produced hydrogen is stored rather than transported to the reactor unit 130. The produced oxygen can be stored or placed back in the atmosphere.

The reactor unit 130 can be configured to react the hydrogen from the electrolyzer unit 120 with carbon dioxide (CO₂) to produce synthetic methane (CH₄). This reaction can be carried out as a Sabatier reaction. The Sabatier reaction produces methane and water from a reaction of hydrogen with carbon dioxide at elevated temperatures and pressures in the presence of a metal catalyst. The temperature range can be about 300° C. to about 400° C. The pressure range can be about 3 bar to about 30 bar (e.g., ˜3×10⁵ Pa to ˜3×10⁶ Pa). The carbon dioxide for the Sabatier reaction can come from a carbon dioxide source, such as, but not limited to, extracted carbon dioxide from the atmosphere. An example method to capture carbon dioxide from the atmosphere includes, but is not limited to, direct air capture using chemical sorbents to bind carbon dioxide. Chemical sorbents can be liquid (e.g., potassium hydroxide (KOH) and aqueous amines) or solid (e.g., amine functionalized solids and metal-organic frameworks (MOF)). In addition, example metal catalysts include, but are not limited to, nickel and ruthenium on alumina (e.g., aluminum oxide). The Sabatier reaction can be described by the following exothermic reaction:

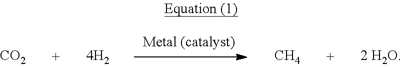

Following the Sabatier reaction, the produced synthetic methane can be received in the storage unit 140 via the gas network 170. The storage unit 140 can be configured to store synthetic methane. For example, the storage unit 140 can be made from materials that are resistant to methane corrosion and leakage. The synthetic methane can be stored on site or be transported and stored off site. In some embodiments, the storage unit 140 is not a battery. The produced water can be removed from the reactor unit 130 by the reactor unit 130 being coupled to the water source or water network 160. In some embodiments, the removed water is recirculated to other parts of the system 100, such as the combustion unit 150 and/or the electrolyzer unit 120.

Synthetic methane can be moved from the storage unit 140 to the combustion unit 150 via the gas network 170. This can be done through a controlled transfer process, which can include pressure regulation, e.g., through pressure regulators, control valves, compressors, and metering. When a deficit of electrical power occurs, the produced or stored synthetic methane can be burned in the combustion unit 150 to generate electricity. Accordingly, the system 100 can meet electrical demands even if there is no source of electrical energy for a period of time by utilizing stored synthetic methane.

The combustion unit 150 can be configured to or provide a suitable environment to burn methane to generate electricity. The combustion of methane is an exothermic reaction where methane reacts with oxygen to produce carbon dioxide, water, and energy. The energy can transfer from the combustion unit 150 as electrical energy to the electric network 180. Oxygen can be sourced from the environment. In addition, produced water can be fed into the water network 160.

The water network 160, the gas network 170, and the electric network 180 can each individually be coupled to more than one unit of the system 100. For example, the water source or water network 160 can be coupled to the electrolyzer unit 120, the reactor unit 130, and the combustion unit 150. This can allow effective and efficient use of water throughout the system 100, as well as water recirculation. The gas network 170 can be coupled to the reactor unit 130, the storage unit 140, and the combustion unit 150. This can allow generated synthetic methane to be used right away or stored for later use. And the electric network 180 can be coupled or reversibly coupled to the electrolyzer unit 120, the reactor unit 130, and the combustion unit 150. This can allow for effective use of energy and generation thereof depending on need.

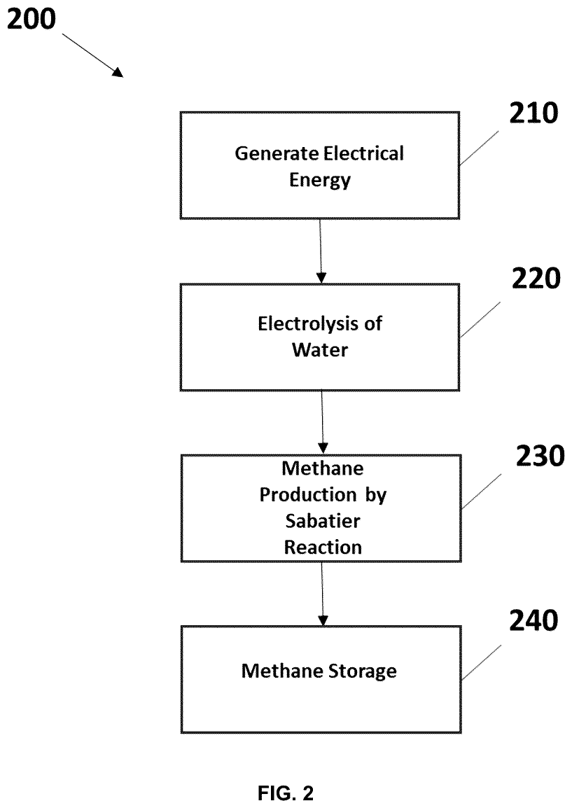

FIG. 2 illustrates an example method 200 for producing synthetic methane. Generally, the method includes, at step 210, generating electrical energy. The electrical energy can be generated from a renewable energy source, such as solar energy. The method further includes, at step 220, electrolyzing water to produce oxygen and hydrogen. The produced hydrogen can then be, at step 230, reacted with carbon dioxide to produce synthetic methane via a Sabatier reaction. The carbon dioxide can be sourced or extracted from the atmosphere. The produced synthetic methane can then be stored, at step 240, or supplied to a natural gas network. The method can also include burning the stored synthetic methane to generate electricity.

The disclosed methods can be implemented using the disclosed systems. For example, electrolyzing water to produce oxygen and hydrogen, at step 220, can be done in an electrolyzer unit 120 configured to electrolyze water to form hydrogen and oxygen as disclosed herein. Likewise, reacting the hydrogen and the carbon dioxide can be done in a reactor unit 130 configured to react the hydrogen from the electrolyzer unit 120 with carbon dioxide to produce synthetic methane in a Sabatier reaction as disclosed herein.

It is understood that the foregoing detailed description and accompanying examples are merely illustrative and are not to be taken as limitations upon the scope of the disclosure. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the technology, may be made without departing from the scope of the disclosure.

For reasons of completeness, the following Embodiments are provided.
Clause 1. A system for producing synthetic methane comprising: a source of electrical energy; an electrolyzer unit electrically coupled to the source of electrical energy, the electrolyzer unit configured to electrolyze water to form hydrogen and oxygen; a source of carbon dioxide; and a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction.
Clause 2. The system of clause 1, comprising a storage unit coupled to the reactor unit, the storage unit configured to store synthetic methane produced by the reactor unit.
Clause 3. The system of clause 1 or 2, wherein the source of electrical energy is generated from solar energy.
Clause 4. The system of any one of clauses 1-3, wherein the source of electrical energy is a solar panel.
Clause 5. The system of any one of clauses 2-4, comprising a combustion unit coupled to the storage unit and the reactor unit via a gas network, the combustion unit configured to burn methane to generate electricity.
Clause 6. The system of clause 5, comprising a water network coupled to the electrolyzer unit, the reactor unit, and the combustion unit.
Clause 7. The system of any one of clauses 1-6, wherein the source of carbon dioxide is the atmosphere.
Clause 8. The system of any one of clauses 5-7, comprising an electric network coupled to the electrolyzer unit, the reactor unit, and the combustion unit.
Clause 9. A system for producing synthetic methane comprising: a source of electrical energy; an electrolyzer unit electrically coupled to the source of electrical energy, the electrolyzer unit configured to electrolyze water to form hydrogen and oxygen; a source of carbon dioxide; a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction; a storage unit coupled to the reactor unit via a gas network, the storage unit configured to store synthetic methane produced by the reactor unit; and a combustion unit coupled to the storage unit and the reactor unit via the gas network, the combustion unit configured to burn methane to generate electricity.
Clause 10. The system of clause 9, comprising a water network coupled to the electrolyzer unit, the reactor unit, and the combustion unit.
Clause 11. A method for producing synthetic methane, the method comprising: using an electrical energy to electrolyze water to produce hydrogen and oxygen; and reacting the hydrogen with carbon dioxide to produce synthetic methane in a Sabatier reaction.
Clause 12. The method of clause 11, further comprising storing the synthetic methane.
Clause 13. The method of clause 11 or 12, further comprising burning the synthetic methane to generate electricity.
Clause 14. The method of any one of clauses 11-13, wherein the electrical energy is generated from solar energy.
Clause 15. The method of any one of clauses 11-14, wherein the electrical energy is generated from a solar panel.
Clause 16. The method of any one of clauses 11-15, wherein the carbon dioxide is extracted from the atmosphere.
Clause 17. The method of any one of clauses 11-16, wherein electrolyzing the water is done in an electrolyzer unit configured to electrolyze water to form hydrogen and oxygen.
Clause 18. The method of clause 17, wherein reacting the hydrogen and the carbon dioxide is done in a reactor unit configured to react the hydrogen from the electrolyzer unit with carbon dioxide to produce synthetic methane in a Sabatier reaction.
Clause 19. The method of any one of clauses 13-18, wherein burning the synthetic methane is done in a combustion unit coupled to the reactor unit, the combustion unit configured to burn methane to generate electricity.
Clause 20. The method of any one of clauses 11-19, wherein the method is carbon neutral.

What is claimed is:

1. A system for producing synthetic methane comprising:
a source of electrical energy;
an electrolyzer unit electrically coupled to the source of electrical energy, the electrolyzer unit configured to electrolyze water to form hydrogen and oxygen;
a source of carbon dioxide; and
a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction.

2. The system of claim 1, comprising a storage unit coupled to the reactor unit, the storage unit configured to store synthetic methane produced by the reactor unit.

3. The system of claim 1, wherein the source of electrical energy is generated from solar energy.

4. The system of claim 1, wherein the source of electrical energy is a solar panel.

5. The system of claim 2, comprising a combustion unit coupled to the storage unit and the reactor unit via a gas network, the combustion unit configured to burn methane to generate electricity.

6. The system of claim 5, comprising a water network coupled to the electrolyzer unit, the reactor unit, and the combustion unit.

7. The system of claim 1, wherein the source of carbon dioxide is the atmosphere.

8. The system of claim 5, comprising an electric network coupled to the electrolyzer unit, the reactor unit, and the combustion unit.

9. A system for producing synthetic methane comprising:
a source of electrical energy;
an electrolyzer unit electrically coupled to the source of electrical energy, the electrolyzer unit configured to electrolyze water to form hydrogen and oxygen;
a source of carbon dioxide;
a reactor unit coupled to the electrolyzer unit and the source of carbon dioxide, the reactor unit configured to react the hydrogen from the electrolyzer unit and the carbon dioxide from the carbon dioxide source to produce synthetic methane in a Sabatier reaction;
a storage unit coupled to the reactor unit via a gas network, the storage unit configured to store synthetic methane produced by the reactor unit; and
a combustion unit coupled to the storage unit and the reactor unit via the gas network, the combustion unit configured to burn methane to generate electricity.

10. The system of claim 9, comprising a water network coupled to the electrolyzer unit, the reactor unit, and the combustion unit.

11. A method for producing synthetic methane, the method comprising:
using an electrical energy to electrolyze water to produce hydrogen and oxygen; and
reacting the hydrogen with carbon dioxide to produce synthetic methane in a Sabatier reaction.

12. The method of claim 11, further comprising storing the synthetic methane.

13. The method of claim 11, further comprising burning the synthetic methane to generate electricity.

14. The method of claim 11, wherein the electrical energy is generated from solar energy.

15. The method of claim 11 wherein the electrical energy is generated from a solar panel.

16. The method of claim 11, wherein the carbon dioxide is extracted from the atmosphere.

17. The method of claim 11, wherein electrolyzing the water is done in an electrolyzer unit configured to electrolyze water to form hydrogen and oxygen.

18. The method of claim 17, wherein reacting the hydrogen and the carbon dioxide is done in a reactor unit configured to react the hydrogen from the electrolyzer unit with carbon dioxide to produce synthetic methane in a Sabatier reaction.

19. The method of claim 13, wherein burning the synthetic methane is done in a combustion unit coupled to the reactor unit, the combustion unit configured to burn methane to generate electricity.

20. The method of claim 11, wherein the method is carbon neutral.
